(19) United States
(12) Patent Application Publication
(10) Pub. No.: US 2024/0188864 A1
(43) Pub. Date: Jun. 13, 2024

(54) EMBEDDED FEATURE FOR IRLEN SYNDROME AUTOMATIC DIAGNOSTIC AND TAILORED SOLUTION

(71) Applicant: Dell Products L.P., Round Rock, TX (US)

(72) Inventors: Marina Fekry Megally Bastarous, Cairo (EG); Ohad Arnon, Beit Nir (IL); Dany Shapiro, Alfei Menashe (IL)

(21) Appl. No.: 18/062,791

(22) Filed: Dec. 7, 2022

(51) Int. Cl. A61B 5/16; A61B 3/14; A61B 5/00; A61M 21/00; G06T 11/00; G06V 40/18

(52) U.S. Cl. CPC A61B 5/163; A61B 3/14; A61B 5/4836; A61M 21/00; G06T 11/001; G06V 40/193; A61M 2021/005; G06T 2210/41

(57) ABSTRACT

An example system includes a camera and a display operable to display text to a human. The system is operable to perform a method that includes tracking, with the camera, eye movements of a human as the human reads text presented by the display, collecting eye movement data, analyzing the eye movement data, based on the analyzing, determining whether or not Irlen Syndrome is indicated by the eye movement data, and when Irlen Syndrome is indicated, adjusting a parameter of the display.

### FIELD OF THE INVENTION

Embodiments of the present invention generally relate to medical diagnostics. More particularly, at least some embodiments of the invention relate to systems, hardware, software, computer-readable media, and methods, for identifying, and compensating for, Irlen Syndrome.

### BACKGROUND

Irlen Syndrome, sometimes referred to as scotopic sensitivity syndrome (SSS), or Meares-Irlen Syndrome, is a disorder that is believed to affect about 12-14% of the population. As presently understood, the brain of individuals with Irlen Syndrome overreacts, or gets overstimulated, from certain visible wavelengths, which may cause physical, cognitive, and neurological problems. One of these problems is reading difficulty. Some individuals have been able to overcome this problem, to varying degrees, by wearing special glasses that include colored filters, by putting a colored filter on top of the hardcopy document they wish to read, or by reading text that is printed on colored paper instead of on white paper.

### BRIEF DESCRIPTION OF THE DRAWINGS

In order to describe the manner in which at least some of the advantages and features of the invention may be obtained, a more particular description of embodiments of the invention will be rendered by reference to specific embodiments thereof which are illustrated in the appended drawings. Understanding that these drawings depict only typical embodiments of the invention and are not therefore to be considered to be limiting of its scope, embodiments of the invention will be described and explained with additional specificity and detail through the use of the accompanying drawings.

FIG. 1 discloses aspects of a system according to one embodiment.

FIG. 2 discloses an example of eye movement tracking according to one embodiment.

FIG. 3 discloses an example of eye movements associated with the presence of Irlen Syndrome.

FIG. 4 discloses an example of eye movements in a subject where Irlen Syndrome is not presented.

FIGS. 5a, 5b, and 5c, disclose example text/background color schemes (see also, the Appendix hereto).

FIG. 6 discloses a method according to one embodiment.

FIG. 7 discloses an example computing entity operable to perform any of the disclosed methods, processes, and operations.

### DETAILED DESCRIPTION OF SOME EXAMPLE EMBODIMENTS

Embodiments of the present invention generally relate to medical diagnostics. More particularly, at least some embodiments of the invention relate to systems, hardware, software, computer-readable media, and methods, for identifying, and compensating for, Irlen Syndrome.

In general, an embodiment of the invention may comprise a method by way of which Irlen Syndrome may be detected through tracking the eye movement of a subject who is reading. The eye movement tracking may be performed, for example, with the use of a camera such as may be included in a personal computer (PC), mobile phone, and various other types of computing devices. The camera may comprise an element of an eye tracker according to one embodiment. An embodiment may also comprise a pattern stability analyzer (PSA) that may generally operate to analyze eye movements obtained by the eye tracker. A color adjuster according to one embodiment may receive the output of the PSA and may use that output to adjust the background color of a screen that displays text to the user. In this way, a user with Irlen Syndrome may be able to accurately read what is displayed on the screen. In an embodiment, the operations of the eye tracker, PSA, and color adjuster may be performed automatically, possibly without the user being aware that such operations are being performed. The eye tracker, PSA, and color adjuster, may each comprise a respective module in one embodiment.

Embodiments of the invention, such as the examples disclosed herein, may be beneficial in a variety of respects. For example, and as will be apparent from the present disclosure, one or more embodiments of the invention may provide one or more advantageous and unexpected effects, in any combination, some examples of which are set forth below. It should be noted that such effects are neither intended, nor should be construed, to limit the scope of the claimed invention in any way. It should further be noted that nothing herein should be construed as constituting an essential or indispensable element of any invention or embodiment. Rather, various aspects of the disclosed embodiments may be combined in a variety of ways so as to define yet further embodiments. For example, any element(s) of any embodiment may be combined with any element(s) of any other embodiment, to define still further embodiments. Such further embodiments are considered as being within the scope of this disclosure. As well, none of the embodiments embraced within the scope of this disclosure should be construed as resolving, or being limited to the resolution of, any particular problem(s). Nor should any such embodiments be construed to implement, or be limited to implementation of, any particular technical effect(s) or solution(s). Finally, it is not required that any embodiment implement any of the advantageous and unexpected effects disclosed herein.

In particular, an embodiment of the invention may automatically diagnose, and compensate for, Irlen Syndrome in a user. As another example, an embodiment may enable a user with Irlen Syndrome to read text displayed on a computing device more effectively than would be possible absent the functionality of such an embodiment. An embodiment may produce more accurate Irlen Syndrome diagnoses than are achieved with diagnostic procedures performed by humans. An embodiment may expand and improve the functionality and operation of computing devices so that such computing devices may operate to improve the visual experience and reading capabilities for those having Irlen Syndrome. As another example, the impacted portion of the population (12-14%) may greatly benefit from an embodiment by improving their educational capabilities and increasing their probability of success in endeavors related to their reading ability, comprehension, and retention. As a final example, an embodiment may operate to diagnose Irlen Syndrome without requiring a user to consciously provide input for analysis. Various other advantages of some example embodiments will be apparent from this disclosure.

It is noted that embodiments of the invention, whether claimed or not, cannot be performed, practically or otherwise, in the mind of a human. Accordingly, nothing herein should be construed as teaching or suggesting that any aspect of any embodiment of the invention could or would be performed, practically or otherwise, in the mind of a human. Further, and unless explicitly indicated otherwise herein, the disclosed methods, processes, and operations, are contemplated as being implemented by computing systems that may comprise hardware and/or software. That is, such methods processes, and operations, are defined as being computer-implemented.

### A. Overview

Irlen Syndrome, also sometimes variously referred to scotopic sensitivity syndrome (SSS), Meares-Irlen Syndrome, or asfedia, is a postulated disorder of vision or image-processing in the brain. The brains of individuals with Irlen Syndrome cannot process certain wavelengths of light, that is, wavelengths which are in the “visible spectrum.” The visible spectrum may range from about 380 nanometers to about 750 nanometers or, expressed in terms of frequency, the visible spectrum may span a band of about 400 terahertz to about 790 terahertz. The visible spectrum may vary somewhat from one person to another.

In individuals with Irlen Syndrome, the brain becomes overstimulated with these wavelengths, and this overstimulation may cause different physical, cognitive, and neurological symptoms in the individual. These symptoms may include difficulty in reading and, correspondingly, comprehension, and retention, of information presented to the user by displayed text. Irlen Syndrome is a relatively common syndrome that is believed to affect about 12-14% of the population. Irlen Syndrome is also believed to affect about 50% of those suffering from other reading disabilities such dyslexia, and about 30% of individuals suffering from ADHD.

Reading difficulties stemming from Irlen Syndrome cannot be explained by phonetic deficits, or by weak vision in the individual. Problems most often occur under conditions of bright lighting, fluorescent lighting, and black/white contrast. For individuals with Irlen Syndrome, text or print may not be clear, stable, or comfortable to look at. As a result, reading can be slow and inefficient, leading to skipping of words or lines, rereading, poor comprehension, and poor or incomplete retention resulting from the lack of comprehension.

Individuals with Irlen Syndrome may be able to read better if they read from colored paper, put tailored colored transparent filters on top of what they are reading, or wear glasses incorporating such filters. These filters may operate to filter out the wavelengths that cause difficulty in reading for the user. Each person may have a color filter or filters combinations that work best for him. During a manual diagnostic procedure, an individual may try different filters, or filter combinations, until that individual finds what works best. This manual diagnostic procedure, which relies heavily on input consciously provided by the test subject, may be referred to as the “Irlen method.”

In contrast with such manual diagnostic, and treatment, approaches, an embodiment may comprise a method that automatically detects whether a user is experiencing difficulty, caused by Irlen Syndrome, with reading text displayed by a computer or other device. After detection of Irlen Syndrome, the embodiment may automatically adjust the screen background color of the text, and/or make other adjustments to the display, that are best suited to the identified needs of the user, so as to possibly enable the user to read better than would be possible using methods such as the manual Irlen method.

A method according to one embodiment does not require a user to consciously provide input to guide the diagnosis, and treatment. Rather, the user input is provided unconsciously, or reflexively. That is, the user may not be consciously aware that he is providing the input, and such input may be provided automatically in response to displayed text visually perceived by the user. Put another way, an embodiment may not require a user to consciously provide affirmative input concerning text that is displayed to the user. User input that may be provided unconsciously, or reflexively, may comprise, for example, eye movements. Thus, in one embodiment, the user input that is provided may comprise, or consist of, reflexive user input.

### B. General Aspects of Some Example Embodiments

Example embodiments of the invention may possess various features and attributes. For example, an embodiment may provide Irlen Syndrome diagnoses that are more accurate than those that are obtained by humans using a manual process. Particularly, a human implemented process may generate a diagnosis by the trial and error addition of text overlay filters, coupled with user input, that is, by asking impacted individuals if they can read better with one set of one or more filters, or another set, where the sets may be compared with each other, and/or with an unfiltered configuration. In such an approach however, the test subject is likely unaware of how a person without Irlen Syndrome is able to read. Thus, the answers provided by the subject may not accurately reflect the reality, and whether or not the subject can read better with the filter(s) in place. That is, it may be difficult for the subject to define and determine whether his ability to read is ‘better’ with the filter(s). As a result of such a process, the color filter selection is suboptimal as compared with results that may be obtained by an embodiment of the invention.

An embodiment of the invention may also provide an approach that is relatively practical, and inexpensive, to implement. By way of contrast, one approach that might be employed for individuals who were diagnosed with the syndrome, is to add an overlay filter over what a person is reading, and another approach may be to used specialized glasses that filter out specific wavelengths known to aggravate Irlen Syndrome. However, such specialized glasses may be expensive and thus unaffordable for some, and are of little use if the user misplaces them so that they are not available when needed. The alternative of adding an overlay filter on top of the laptop screen is not user friendly, is not adaptable to changing conditions in the user environment and/or in the user, and is not configured to readily adjust to different screen sizes, such as a laptop and mobile phone for example. In contrast, an embodiment may be implemented in the user device itself, thus obviating the need for filter screens. As well, an embodiment may automatically adjust to, among other things, (1) changing conditions in the user environment such as, for example, different types and intensities of light, and (2) changes in the user anatomy such as, for example, changes in the number, frequency, and magnitude, of eye movements.

As another example of the features and attributes of some embodiments of the invention, and as alluded to earlier herein, an embodiment may be adaptable to changing conditions in an operating environment, and may not be limited only to specific-based, or environment-based, approaches. Particularly, and by way of contrast with one embodiment of the invention, an approach may be implemented in which a diagnosis may be performed under a specific, constrained, set of conditions. For example, the diagnosis may be performed with a certain type of light, at a particular time of day, and for a user with specific physical conditions. Thus, and in contrast with an embodiment of the invention, such an approach is limited to a specific situation and environmental conditions, and is not able to readily adapt to changing situations and conditions. This is problematic inasmuch as studies show that different conditions influence the optimal filters that would be selected for a particular individual, meaning that the required filter will change based on different conditions during the day. As a result, although the individual would be provided with a specific filter that suits his test conditions, he may need to use different filters if the situation and environmental conditions were to change.

As a final example of some features and attributes of some embodiments of the invention, an embodiment of the invention may be well suited to provide diagnoses of Irlen Syndrome in children and, as such, an embodiment may help improve the pace and thoroughness of the education of children. By way of background, where children are concerned, the lack of diagnosis of Irlen syndrome may have several causes.

For example, parents or teachers must notice that the child suffers from learning challenges before a diagnosis can be attempted. Oftentimes however, the cause for such challenges escapes the notice of parents and teachers who may simply think that if the child works harder, he can do better.

As another example, once a child is diagnosed with learning challenges that are not caused by a lack of motivation, the child must go through a long and expensive diagnostic process to find the root cause behind his learning challenges. When it comes diagnosis of adults, the problem of obtaining an accurate diagnosis may be more difficult, as an adult may have acquired low self-esteem caused by the inability to acquire good reading ability since childhood. An adult may thus be unlikely to seek a diagnosis since he believes that nothing can change his situation.

The failure to obtain an Irlen Syndrome diagnosis may cause individuals to simply give up on the acquisition of adequate reading capabilities. The lack of such capabilities may in turn impair the ability of the individual to acquire further education later.

### C. Detailed Description of Some Example Embodiments

### C.1 Example System and Operations

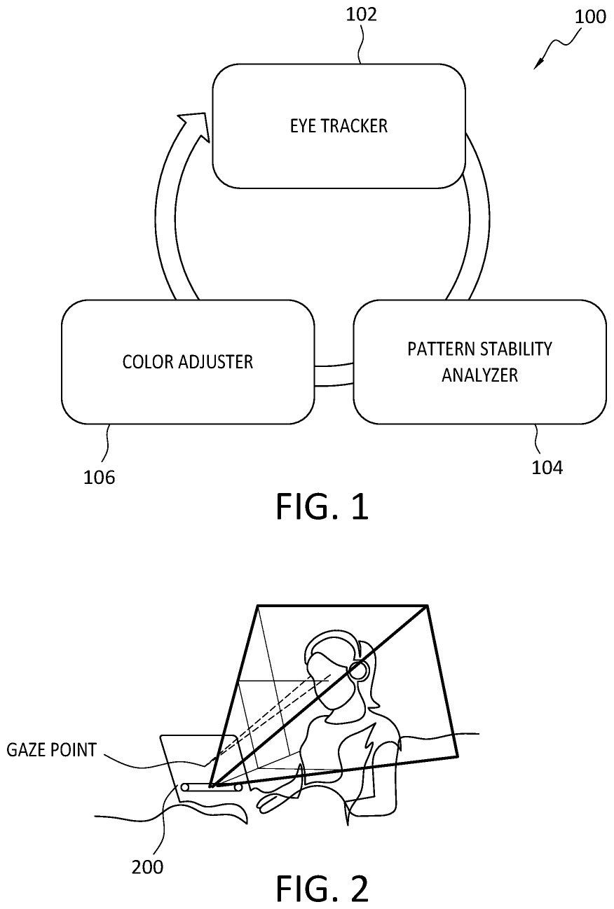

One or more embodiments of the invention may comprise various different components, each of which may comprise hardware and/or software. With reference to FIG. 1, a system 100 according to an embodiment is disclosed. The system 100 may be partly, or completely, integrated into, or with, any device that comprises (1) a display configured and arranged to display text to a user, and (2) a camera or other equipment configured and arranged to capture still images and/or video of a user of the device. Such devices may include, but are not limited to, laptop computers, desktop computers, electronic notebooks (such as the Apple iPad® for example), electronic reading devices (such as the Amazon Kindle® for example), e-books (such as the Barnes and Noble Nook® for example), and mobile phones, for example. In an embodiment, the system 100 may be configured as a pluggable device, which may comprise a USB (uniform serial bus) connection, that can be connected to a host device, examples of which are noted above. In this way, the system 100 may be employed with a host device that lacks an on-board camera.

The system 100 may comprise an eye tracker 102 that may be operable to use a camera, such as a camera in a laptop for example, to track, possibly in real time, the eye movements of a user. These eye movements may be collected as data for analysis, as discussed elsewhere herein. Among other things, the eye tracker 102 may operate, in conjunction with a camera, to observe user viewing patterns and study what users are looking at, and how the user gaze point moves to different positions on a display 200, as shown FIG. 2. An embodiment of the eye tracker 102 may be configured, and operate, according to various approaches, examples of which are disclosed in “Eye tracking for spatial research: Cognition, computation, challenges” (//irlen.com/), which is incorporated herein in its entirety by this reference.

It is noted that the eye tracker 102 may, in addition to gathering user eye movement data, also gather, or direct the gathering of, information about the ambient lighting where the user is located, and -information about the display screen, such as the color and brightness of the displayed text and the background against which the text is displayed to the user. In an embodiment, these data regarding ambient lighting and the display screen may be gathered by a camera, and/or other components, and passed to the eye tracker 102, and/or to other components of the system 100.

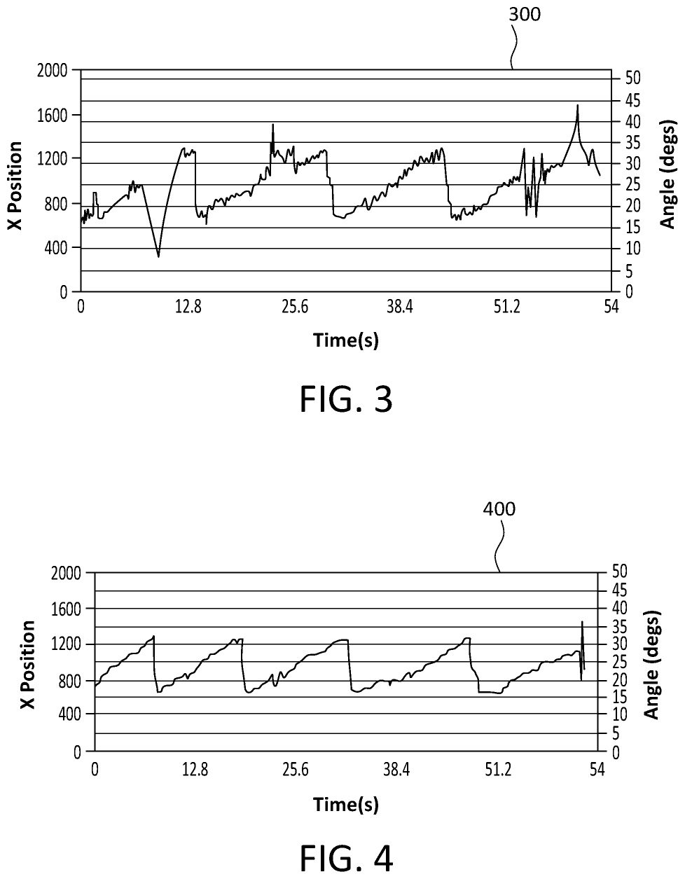

With continued reference to FIG. 1, the system 100 may comprise a pattern stability analyzer (PSA) 104 that may receive, as input, data gathered by and/or at the direction of the eye tracker 102. Among other things, the PSA 104 may be programmed to analyze the pattern of movements of one or both of the eyes of the user, as well as the gaze point stability, and then determine if the user eye movements fit within the bounds of normal behavior, or if the user eye movements are unstable to the extent that would be expected in people with Irlen Syndrome. As to the latter, for example, a user with Irlen Syndrome may skip words, skip lines, reread, and/or move his eyes laterally more than a user that does not have Irlen Syndrome. In this regard, see FIG. 3 which discloses a graph 300 that shows eye movements of a user with Irlen Syndrome, and FIG. 4, which discloses a graph 400 that shows eye movements of a user that does not have Irlen Syndrome, that is, normal eye movements. As shown in FIG. 3, the eye movements of a user with Irlen Syndrome may be somewhat erratic and random, while the eye movements of a user without Irlen Syndrome, shown in FIG. 4, may be relatively consistent and predictable.

As shown in FIG. 1, the system 100 may comprise a color adjuster 106 that may be configured to communicate with the eye tracker 102, and the PSA 104. In an embodiment, the color adjuster 106 may operate to adjust the respective color backgrounds for one or more application to a color taken from a set of predefined colors that have been determined to alleviate Irlen Syndrome effects for a particular user. In more detail, once the PSA 104 detects that the eye movements and the gaze point stability are similar to those of people not having Irlen Syndrome, the color adjuster 106 may stop changing the background color, and the system 100 may transition from a learning phase into a monitoring phase in which the eye tracker 102 may continue to monitor user eye behavior. If an aspect of eye behavior, such as eye movement, is determined by the PSA 104 to be outside the bounds of normal eye behavior, the PSA 104 may signal the color adjuster 106 to change the background color accordingly, and then compare the eye movement resulting from the change in color with normal eye movement to determine if further color adjustments are needed.

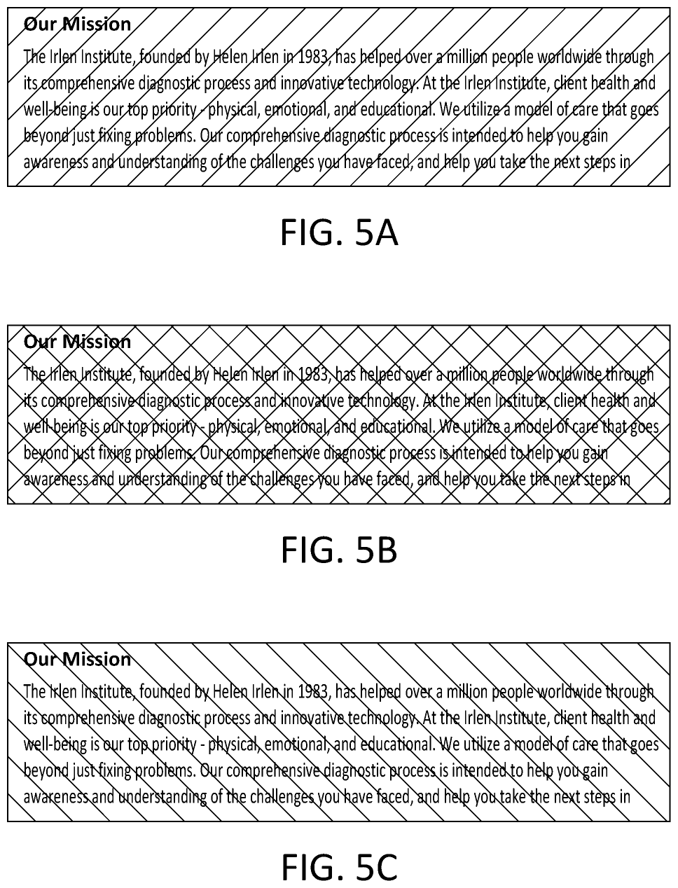

The operations in this feedback loop may be performed on an ongoing basis in the background while the user reads the text. As such, a user may be unaware that color adjustments are being made to his display. Some examples of text/background color combinations are disclosed in gray-scale in FIGS. 5a-5c, and in color in the Appendix hereto, which is incorporated into this disclosure in its entirety by this reference.

### C.2 Further Example Operations of an Embodiment

It is noted that the eye tracker 102 may collect eye movement data at least until such time as the system 100 is able to provide (1) a diagnosis as to whether or not Irlen Syndrome is presented, and (2) if so, a user-specific solution. In an embodiment, and possibly as part of a monitoring process, eye movement data may be collected on an ongoing basis in circumstances where changes in the environment of the user, and/or changes regarding the eyes of the user, are possible or expected. This data collection, and consequent automatic adjustments of the user display, may be performed automatically, and on-the-fly, as user conditions and environment conditions change. Thus, for example, the system 100 may be able to automatically adjust one or more attributes of a device display, such as text color, background color, and brightness for example, as needed to suit the needs of a particular user, and to accommodate any changes in the physical environment of the user, such as changes in the type and intensity of lighting.

As noted, in an embodiment, data collection may be ongoing. Further, in an embodiment, data collection may only be performed during one or more discrete timeframes whose respective boundaries may be automatically defined by the system 100, and/or by a user. In an embodiment, a timeframe may, or may not, correspond, for example, to a time during which the user is experiencing a particular physiological condition.

It is noted further that various physiological conditions, which may be permanent or transient, of a user may affect the eye movements of that user. For example, if the user is tired, under stress, or ill, the eye movements of the user may be affected. An embodiment of the invention may account for such physiological conditions by automatically adjusting the user display in response to eye movements that may be characteristic of such physiological conditions.

An embodiment of the invention may operate using a 32-bit color palette, which may comprise about 16.5 million colors. In contrast, manual approaches to Irlen Syndrome diagnoses may use only about 12 colors. Thus, an embodiment may be able to finely tune the colors of a display to suit a particular user by using such a highly granular color palette.

### D. Further Discussion

Some embodiments of the invention may possess various useful features and advantages. A non-exhaustive listing of some of such features and advantages is presented below.

For example, an embodiment may comprise an eye tracking that detects instability in eye position and eye movements of an individual. The eye movement information collected by an embodiment of the invention may not be affected or influenced by subjective feedback provided by the individual. In an embodiment, only data reflexively provided by a user is employed, and any input consciously provided by a user may be ignored or unutilized by an embodiment. This feature may be particularly useful where children are concerned, as they may not be able to fully or accurately describe what they are experiencing.

In a digital diagnostic procedure according to an embodiment, a system may be configured and employed that is sensitive to minor changes of the eye movements, and the system may operate to find the background color that best helps a person to read, by comparing the eye movement pattern resulted using different text background colors. As such, the accuracy of a diagnostic digital procedure according to an embodiment may be materially higher than results obtained through use of a human, or manual, diagnostic. That is, an embodiment of the invention may provide a more accurate diagnosis, and resolution, of Irlen Syndrome in a user, than could be obtained with a manual diagnostic approach conducted by a human, and based on input solicited from, and consciously provided by, a test subject.

As another example of a feature or advantage, an embodiment may enable ready adjustment of the color of a screen background without the need for extra accessories or equipment. For example, an embodiment may be configured so that no extra accessories are needed to filter out the unwanted wavelength(s), that is, wavelengths that may aggravate the effects of, or trigger, Irlen Syndrome. In an embodiment, a device such as a computer may implement a method that automatically changes the background color of a display, which may provide nearly immediate relief for a user suffering from Irlen syndrome. In this way, an embodiment may implement a solution that, as compared with manual diagnostic approaches and solutions, is simpler, cheaper, and accessible to anyone using a device for the visual display of text.

An embodiment may enable the screen background color to be changed automatically to provide the best vision, for a given user, at the different environmental conditions whenever needed. Since the eye movements may be monitored, by an eye tracking component, at all times while a user is viewing a display, a diagnostic and display adjustment method according to one embodiment may be executed continually in a short period, for a longer period, or for a period with no defined end. In addition, in an embodiment, the color spectrum of the display may be very wide, so as to enable an embodiment to dynamically change the display color to accommodate a specific transient condition in the eyes of the user, and/or in an environment in which the user is viewing a display. Thus, an embodiment may operate to identify an optimum, user-specific, screen background color that enhances the vision of the user under various different external conditions which may include, but are not limited to, bright lighting, fluorescent lighting, black/white contrast, current time of day, and to the individual physiological conditions that may stem from tiredness, or illness, for example, and which may affect eye movements.

As a final example, an embodiment may automatically diagnose Irlen syndrome by tracking eye movement while reading using a PC or other device with a display that is capable of visually presenting text to a user. By embedding an embodiment in a computing device, Irlen syndrome diagnostic capability will accessible for anyone using that device. Because an embodiment may detect, through the eye movement patterns, whether a user has Irlen syndrome or not, a computing device that implements an embodiment of the invention may be usable both by users without Irlen Syndrome, and by users who have Irlen Syndrome. Moreover, an embodiment may automatically change the screen background color for a user, without requiring any user action, so that his reading ability may be optimized. In this way, an embodiment may automatically improve the reading ability for those who suffer from Irlen syndrome, and thus possibly improve their chance to acquire education.

### E. An Example Use Case

One non-limiting use case concerns an individual who is using his PC or laptop. In this case, an embodiment may constantly monitor the eye movement of the individual, and compare the detected eye movement to the expected normal behavioral. Once the system detects that the eye movement is not as expected, such as which may occur when Irlen syndrome is presented, the system may suggest, to the user, that the background color should be changed to a particular new color.

For each different background color, an embodiment may continue to monitor the eye movement, and once the monitored eye movement pattern is similar to the expected pattern, the system will notify the user that the current color is optimal for him. When the user is using the optimal background color, the system may transition to a monitoring stage, during which the system may continue to monitor the eye movement of the individual, and adjust the background color again if needed. In this way, an embodiment may improve the ability of a user to read text displayed by a device.

### F. Example Methods

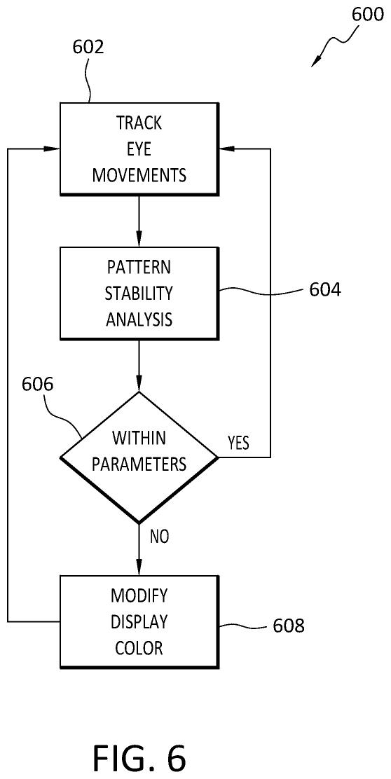

It is noted with respect to the disclosed methods, including the example method of FIG. 6, that any operation(s) of any of these methods, may be performed in response to, as a result of, and/or, based upon, the performance of any preceding operation(s). Correspondingly, performance of one or more operations, for example, may be a predicate or trigger to subsequent performance of one or more additional operations. Thus, for example, the various operations that may make up a method may be linked together or otherwise associated with each other by way of relations such as the examples just noted. Finally, and while it is not required, the individual operations that make up the various example methods disclosed herein are, in some embodiments, performed in the specific sequence recited in those examples. In other embodiments, the individual operations that make up a disclosed method may be performed in a sequence other than the specific sequence recited.

Directing attention now to FIG. 6, an example method according to one embodiment is denoted generally at 600. The method 600 may be performed in whole, or in part, by a device, such as a computer or mobile phone for example, that includes a display operable to display text to a user. The display may be modifiable so that attributes such as the color and intensity of text and/or the background behind the text may be modified as part of the operation of an embodiment. The device may include a UI that may enable a user to move from one part of the text to another part of the text, such as by “turning” electronic pages, or scrolling, for example.

The example method 600 may begin with the tracking 602 of the eye movements of a user. This tracking may be performed using a camera. The eye movement information collected by the tracking 602 may then be provided to a pattern stability analyzer which may perform a pattern stability analysis 604, and/or other analyses, of the eye movement information that has been collected.

After the pattern stability analysis 604 has been performed, a determination 606 may be made, based on the outcome of the pattern stability analysis 604, as to whether or not the eye movements of the user fall within a defined range, such as a “normal” range or, alternatively, are indicative of Irlen Syndrome. In the former case, there may be no adjustment needed to the user display, and the method 600 may enter a monitoring phase by returning to 602. In the latter case, the system may adjust one or more display parameters 608, such as color and intensity for example. The adjustments may be made to the text and/or to the background on which the text is displayed.

After these adjustments have been made, the method 600 may return to 602 to gather information about how the adjustments may have affected the eye movements. This loop may continue until the eye movements are determined 606 to be with parameters, at which point the method 600 may enter a monitoring phase by returning to 602.

### G. Further Example Embodiments

Following are some further example embodiments of the invention. These are presented only by way of example and are not intended to limit the scope of the invention in any way.

Embodiment 1. A method, comprising: tracking, with a camera, eye movements of a human as the human reads text presented by a display; collecting eye movement data; analyzing the eye movement data; based on the analyzing, determining whether or not Irlen Syndrome is indicated by the eye movement data; and when Irlen Syndrome is indicated, adjusting a parameter of the display.

Embodiment 2. The method as recited in embodiment 1, wherein the parameter is adjusted automatically when Irlen Syndrome is indicated.

Embodiment 3. The method as recited in any of embodiments 1-2, wherein the camera and the display are elements of a computing device.

Embodiment 4. The method as recited in any of embodiments 1-3, wherein the parameter of the display is a background color against which the text is displayed.

Embodiment 5. The method as recited in any of embodiments 1-4, wherein the determining is performed without use of any input consciously provided by the human.

Embodiment 6. The method as recited in any of embodiments 1-4, wherein the determining is performed using only input that is reflexively provided by the human.

Embodiment 7. The method as recited in any of embodiments 1-6, wherein the determining that Irlen Syndrome is presented, or not, is performed automatically.

Embodiment 8. A system, comprising hardware and/or software, operable to perform any of the operations, methods, or processes, or any portion of any of these, disclosed herein.

Embodiment 9. A non-transitory storage medium having stored therein instructions that are executable by one or more hardware processors to perform operations comprising the operations of any one or more of embodiments 1-8.

### H. Example Computing Devices and Associated Media

The embodiments disclosed herein may include the use of a special purpose or general-purpose computer including various computer hardware or software modules, as discussed in greater detail below. A computer may include a processor and computer storage media carrying instructions that, when executed by the processor and/or caused to be executed by the processor, perform any one or more of the methods disclosed herein, or any part(s) of any method disclosed.

As indicated above, embodiments within the scope of the present invention also include computer storage media, which are physical media for carrying or having computer-executable instructions or data structures stored thereon. Such computer storage media may be any available physical media that may be accessed by a general purpose or special purpose computer.

By way of example, and not limitation, such computer storage media may comprise hardware storage such as solid state disk/device (SSD), RAM, ROM, EEPROM, CD-ROM, flash memory, phase-change memory (“PCM”), or other optical disk storage, magnetic disk storage or other magnetic storage devices, or any other hardware storage devices which may be used to store program code in the form of computer-executable instructions or data structures, which may be accessed and executed by a general-purpose or special-purpose computer system to implement the disclosed functionality of the invention. Combinations of the above should also be included within the scope of computer storage media. Such media are also examples of non-transitory storage media, and non-transitory storage media also embraces cloud-based storage systems and structures, although the scope of the invention is not limited to these examples of non-transitory storage media.

Computer-executable instructions comprise, for example, instructions and data which, when executed, cause a general purpose computer, special purpose computer, or special purpose processing device to perform a certain function or group of functions. As such, some embodiments of the invention may be downloadable to one or more systems or devices, for example, from a website, mesh topology, or other source. As well, the scope of the invention embraces any hardware system or device that comprises an instance of an application that comprises the disclosed executable instructions.

Although the subject matter has been described in language specific to structural features and/or methodological acts, it is to be understood that the subject matter defined in the appended claims is not necessarily limited to the specific features or acts described above. Rather, the specific features and acts disclosed herein are disclosed as example forms of implementing the claims.

As used herein, the term ‘module’ or ‘component’ may refer to software objects or routines that execute on the computing system. The different components, modules, engines, and services described herein may be implemented as objects or processes that execute on the computing system, for example, as separate threads. While the system and methods described herein may be implemented in software, implementations in hardware or a combination of software and hardware are also possible and contemplated. In the present disclosure, a ‘computing entity’ may be any computing system as previously defined herein, or any module or combination of modules running on a computing system.

In at least some instances, a hardware processor is provided that is operable to carry out executable instructions for performing a method or process, such as the methods and processes disclosed herein. The hardware processor may or may not comprise an element of other hardware, such as the computing devices and systems disclosed herein.

In terms of computing environments, embodiments of the invention may be performed in client-server environments, whether network or local environments, or in any other suitable environment. Suitable operating environments for at least some embodiments of the invention include cloud computing environments where one or more of a client, server, or other machine may reside and operate in a cloud environment.

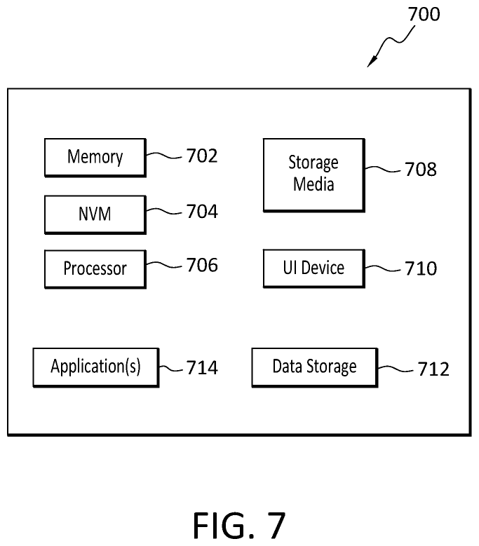

With reference briefly now to FIG. 7, any one or more of the entities disclosed, or implied, by FIGS. 1-6 and/or elsewhere herein, may take the form of, or include, or be implemented on, or hosted by, a physical computing device, one example of which is denoted at 700. As well, where any of the aforementioned elements comprise or consist of a virtual machine (VM), that VM may constitute a virtualization of any combination of the physical components disclosed in FIG. 7.

In the example of FIG. 7, the physical computing device 700 includes a memory 702 which may include one, some, or all, of random access memory (RAM), non-volatile memory (NVM) 704 such as NVRAM for example, read-only memory (ROM), and persistent memory, one or more hardware processors 706, non-transitory storage media 708, UI (user interface) device 710, and data storage 712. One or more of the memory components 702 of the physical computing device 700 may take the form of solid state device (SSD) storage. As well, one or more applications 714 may be provided that comprise instructions executable by one or more hardware processors 706 to perform any of the operations, or portions thereof, disclosed herein.

Such executable instructions may take various forms including, for example, instructions executable to perform any method or portion thereof disclosed herein, and/or executable by/at any of a storage site, whether on-premises at an enterprise, or a cloud computing site, client, datacenter, data protection site including a cloud storage site, or backup server, to perform any of the functions disclosed herein. As well, such instructions may be executable to perform any of the other operations and methods, and any portions thereof, disclosed herein.

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

What is claimed is:

1. A method, comprising:
tracking, with a camera, eye movements of a human as the human reads text presented by a display;
collecting eye movement data;
analyzing the eye movement data;
based on the analyzing, determining whether or not Irlen Syndrome is indicated by the eye movement data; and
when Irlen Syndrome is indicated, adjusting a parameter of the display.

2. The method as recited in claim 1, wherein the parameter is adjusted automatically when Irlen Syndrome is indicated.

3. The method as recited in claim 1, wherein the camera and the display are elements of a computing device.

4. The method as recited in claim 1, wherein the parameter of the display is a background color against which the text is displayed.

5. The method as recited in claim 1, wherein the determining is performed without use of any input consciously provided by the human.

6. The method as recited in claim 1, wherein the determining is performed using only input that is reflexively provided by the human.

7. The method as recited in claim 1, wherein the determining that Irlen Syndrome is presented, or not, is performed automatically.

8. A non-transitory storage medium having stored therein instructions that are executable by one or more hardware processors to perform operations comprising:
tracking, with a camera, eye movements of a human as the human reads text presented by a display;
collecting eye movement data;
analyzing the eye movement data;
based on the analyzing, determining whether or not Irlen Syndrome is indicated by the eye movement data; and
when Irlen Syndrome is indicated, adjusting a parameter of the display.

9. The non-transitory storage medium as recited in claim 8, wherein the parameter is adjusted automatically when Irlen Syndrome is indicated.

10. The non-transitory storage medium as recited in claim 8, wherein the camera and the display are elements of a computing device.

11. The non-transitory storage medium as recited in claim 8, wherein the parameter of the display is a background color against which the text is displayed.

12. The non-transitory storage medium as recited in claim 8, wherein the determining is performed without use of any input consciously provided by the human.

13. The non-transitory storage medium as recited in claim 8, wherein the determining is performed using only input that is reflexively provided by the human.

14. The non-transitory storage medium as recited in claim 8, wherein the determining that Irlen Syndrome is presented, or not, is performed automatically.

15. A system, comprising:
one or more hardware processors; and
a non-transitory storage medium having stored therein instructions that are executable by the one or more hardware processors to perform operations comprising:
tracking, with a camera, eye movements of a human as the human reads text presented by a display;
collecting eye movement data;
analyzing the eye movement data;
based on the analyzing, determining whether or not Irlen Syndrome is indicated by the eye movement data; and
when Irlen Syndrome is indicated, adjusting a parameter of the display.

16. The system as recited in claim 15, wherein the parameter is adjusted automatically when Irlen Syndrome is indicated.

17. The system as recited in claim 15, wherein the system comprises a computing device that includes the camera and the display.

18. The system as recited in claim 15, wherein the parameter of the display is a background color against which the text is displayed.

19. The system as recited in claim 15, wherein the determining is performed using only input that is reflexively provided by the human.

20. The system as recited in claim 15, wherein the determining that Irlen Syndrome is presented, or not, is performed automatically.
